(19) United States
(12) Patent Application Publication
(10) Pub. No.: US 2018/0235649 A1
(43) Pub. Date: Aug. 23, 2018

(54) ADJUSTABLE SURGICAL DEVICE WITH ULTRASOUND GUIDANCE AND RELATED TECHNIQUES

(71) Applicant: Hisham Elkadi, Canton, MA (US)

(72) Inventor: Hisham Elkadi, Canton, MA (US)

(21) Appl. No.: 15/663,988

(22) Filed: Jul. 31, 2017

Related U.S. Application Data
(63) Continuation of application No. PCT/US17/44366, filed on Jul. 28, 2017.
(60) Provisional application No. 62/461,973, filed on Feb. 22, 2017.

(51) Int. Cl. A61B 17/32; A61B 17/28; A61B 18/14; A61B 18/12

(52) U.S. Cl. CPC A61B 17/320068; A61B 17/28; A61B 2017/00734; A61B 18/1445; A61B 18/1206; A61B 17/320092

(57) ABSTRACT

A surgical device with ultrasound guidance and related techniques are disclosed. The disclosed surgical device includes an ultrasound probe and one or more guide portions configured to host a given surgical instrument. A given guide portion may be configured to permit linear movement of a surgical instrument hosted thereby, as well as angular movement of that instrument with respect to the ultrasound probe. In this manner, the disclosed surgical device may be configured to provide improved visualization of the surgical instrument(s), as well as a target tissue mass, by the ultrasound probe. This may realize improvements in control during the surgical process and in the efficiency of tissue removal as compared to existing approaches. The disclosed surgical device may be configured for use in a manner reducing the opportunity for ultrasound interference which otherwise might result in utilizing an ultrasound probe and an ultrasonic surgical instrument simultaneously.

### CROSS-REFERENCE TO RELATED APPLICATIONS

This patent application is a Continuation of PCT International Patent Application No. PCT/US17/44366, titled “Adjustable Surgical Device with Ultrasound Guidance and Related Techniques,” filed on Jul. 28, 2017, which claims the benefit of and priority to U.S. Provisional Patent Application No. 62/461,973, titled “Surgical Device with Ultrasound Guidance,” filed on Feb. 22, 2017. Each of these patent applications is herein incorporated by reference in its entirety.

### FIELD OF THE DISCLOSURE

The present disclosure relates to surgical devices and more particularly to surgical devices including ultrasound guidance.

### BACKGROUND

In electrosurgery, high-frequency electric current is used in cutting and coagulating tissue at a surgical site. In performing the different surgical actions, the current may be varied as desired. Typically, the pathway of the current in operation of the device is dictated by whether the electrosurgical instrument is of monopolar or bipolar type.

### SUMMARY

The subject matter of this application may involve, in some cases, interrelated products, alternative solutions to a particular problem, and/or a plurality of different uses of a single system or article.

One example embodiment provides a surgical device. The surgical device includes an ultrasound probe. The surgical device further includes at least one guide portion including: a tubular body portion having a hollow inner bore defined therein and configured to host a surgical instrument; and a tab portion extending from the body portion and configured to be mounted to an exterior of the ultrasound probe via a retention pin, wherein the tab portion includes a chamfer configured to be incident with an exterior of the ultrasound probe. The at least one guide portion is configured to permit: linear movement of the surgical instrument with respect to the at least one guide portion; and angular movement of the surgical instrument with respect to the ultrasound probe, with the chamfer being configured to provide a stopping point for the angular movement when incident with the exterior of the ultrasound probe. In some cases, the at least one guide portion includes two guide portions, one of which is disposed at a first side of the ultrasound probe, and the other of which is disposed at an opposing second side of the ultrasound probe. In some such cases, the two guide portions are dissimilar in at least one dimension. In some instances, the angular movement of the surgical instrument with respect to the ultrasound probe is planar. In some cases, the angular movement of the surgical instrument with respect to the ultrasound probe is triaxial. In some instances, the tubular body portion is adjustable in at least one dimension. In some cases, the guide portion is of monolithic construction. In some instances, in being configured to host the surgical instrument, the guide portion is configured to host at least one of an electrosurgical knife, an articulated electrosurgical knife, electrosurgical forceps, an ultrasound knife, and ultrasound forceps. In some cases, the surgical device further includes at least one control feature provided on the ultrasound probe and configured to control a surgical modality of the surgical instrument hosted by the guide portion. In some instances, the surgical device further includes at least one of: an integrated battery; and a removable battery. In some cases, the surgical device further includes a display element configured to display imaging data received from the ultrasound probe.

In some instances, a surgical system is provided, including the surgical device and a power supply and control unit configured to be operatively coupled with the surgical device. In some cases, the power supply and control unit is an electrosurgical power supply and control unit. In some cases, the power supply and control unit is an ultrasound power supply and control unit. In some instances, the surgical system further includes a foot pedal control configured to: be operatively coupled with at least one of the surgical device and the power supply and control unit; and control a surgical modality of the surgical instrument hosted by the guide portion of the surgical device. In some cases, the surgical system further includes an ultrasound imaging display device configured to be operatively coupled with the surgical device and to display imaging data received from the ultrasound probe of the surgical device.

Another example embodiment provides a method of reducing ultrasound interference in using a surgical device, the method including: performing, via an ultrasound probe of the surgical device, a first ultrasound scan for a first period; ceasing performing the first ultrasound scan, and operating at least one surgical instrument hosted by the surgical device for a second period; and ceasing operating the at least one surgical instrument, and performing, via the ultrasound probe of the surgical device, a second ultrasound scan for a third period. In some cases, at least one of the first period and the third period is of a duration of 1 sec or less. In some instances, the at least one surgical instrument is hosted by the surgical device via a guide portion including: a tubular body portion having a hollow inner bore defined therein and configured to host the at least one surgical instrument; and a tab portion extending from the body portion and configured to be mounted to an exterior of the ultrasound probe via a retention pin, wherein the tab portion includes a chamfer configured to be incident with an exterior of the ultrasound probe. Additionally, the guide portion is configured to permit: linear movement of the at least one surgical instrument with respect to the guide portion; and angular movement of the at least one surgical instrument with respect to the ultrasound probe, with the chamfer being configured to provide a stopping point for the angular movement when incident with the exterior of the ultrasound probe. In some cases, at least one of performing the first ultrasound scan, ceasing performing the first ultrasound scan, operating the at least one surgical instrument, ceasing operating the at least one surgical instrument, and performing the second ultrasound scan is performed under guidance from a computing element configured to communicate with the surgical device.

The features and advantages described herein are not all-inclusive and, in particular, many additional features and advantages will be apparent to one of ordinary skill in the art in view of the drawings, specification, and claims. Moreover, it should be noted that the language used in the specification has been selected principally for readability and instructional purposes and not to limit the scope of the inventive subject matter.

### BRIEF DESCRIPTION OF THE DRAWINGS

FIG. 1 illustrates an isometric view of a surgical device configured in accordance with an embodiment of the present disclosure.

FIG. 2 illustrates a top-down plan view of the surgical device of FIG. 1.

FIG. 3 illustrates a bottom-up plan view of the surgical device of FIG. 1.

FIG. 4 illustrates an isometric view of a guide portion configured in accordance with an embodiment of the present disclosure.

FIG. 5 illustrates a partially exploded end view of a surgical device including several guide portions, in accordance with an embodiment of the present disclosure.

FIG. 6 illustrates a partial top-down view of a surgical device including several guide portions, in accordance with an embodiment of the present disclosure.

FIG. 7 illustrates an isometric view of a surgical device with an assortment of surgical instruments which it may be configured to host, in accordance with some embodiments of the present disclosure.

FIG. 8 illustrates an isometric view of a straight monopolar electrosurgical knife configured in accordance with an embodiment of the present disclosure.

FIGS. 9A-9B illustrate several isometric views of an articulated monopolar electrosurgical knife configured in accordance with an embodiment of the present disclosure.

FIG. 10 illustrates an isometric view of narrow-grip bipolar electrosurgical forceps configured in accordance with an embodiment of the present disclosure.

FIG. 11 illustrates an isometric view of wide-grip bipolar electrosurgical forceps configured in accordance with an embodiment of the present disclosure.

FIG. 12 illustrates an isometric view of an ultrasound straight knife configured in accordance with an embodiment of the present disclosure.

FIG. 13 illustrates an example surgical system configured in accordance with an embodiment of the present disclosure.

FIG. 14 is a flow chart illustrating a method of reducing ultrasound interference in use of a surgical device, in accordance with an embodiment of the present disclosure.

These and other features of the present embodiments will be understood better by reading the following detailed description, taken together with the figures herein described. In the drawings, each identical or nearly identical component that is illustrated in various figures may be represented by a like numeral. For purposes of clarity, not every component may be labeled in every drawing. Furthermore, as will be appreciated in light of this disclosure, the accompanying drawings are not intended to be drawn to scale or to limit the described embodiments to the specific configurations shown.

### DETAILED DESCRIPTION

### General Overview

In accordance with some embodiments of the present disclosure, a surgical device with ultrasound guidance and related techniques are disclosed. The disclosed surgical device includes an ultrasound probe and one or more guide portions configured to host a given surgical instrument. A given guide portion may be configured, in accordance with some embodiments, to permit linear movement of a surgical instrument hosted thereby, as well as angular movement of that instrument with respect to the ultrasound probe. In this manner, the disclosed surgical device may be configured to provide improved visualization of the surgical instrument(s), as well as a target tissue mass, by the ultrasound probe. This may realize improvements in control during the surgical process and in the efficiency of tissue removal as compared to existing approaches. In accordance with some embodiments, the disclosed surgical device may be configured for use in a manner reducing the opportunity for ultrasound interference which otherwise might result in utilizing an ultrasound probe and an ultrasonic surgical instrument simultaneously. Numerous configurations and variations will be apparent in light of this disclosure.

In accordance with some embodiments, a surgical device configured as provided herein may be utilized, for example, in excising tissue from a patient during a surgical procedure. Generally, the disclosed surgical device may be configured, in accordance with some embodiments, for use in tumor or other target tissue mass removal procedures. In an example case, the surgical device may be configured for use, for instance, in breast mass biopsy procedures. To these various ends, the ultrasound probe may assist the physician or other user in detecting the entire target tissue mass and in guiding the device to the proper location for performing any of the available surgical actions. In some embodiments, a given guide portion may be adjustable in one or more directions to maximize or otherwise customize visualization by the ultrasound probe of a surgical instrument hosted by that guide portion. As will be appreciated in light of this disclosure, such adjustability also may facilitate removal of the entire target tissue mass, at least in some instances.

In accordance with some embodiments, the disclosed surgical device may be utilized in any of a wide range of surgical procedures and contexts, including, for example: (1) breast mass excision, optionally with needle localization; (2) superficial breast mass excision; (3) re-excision of a breast mass; and (4) subcutaneous mass excision, including lipoma, lymph node biopsy, melanoma excision, and inclusion cyst. In accordance with some embodiments, the disclosed surgical device may be utilized in pain management, including radio-frequency ablation (RFA) of the facet joint and sacroiliac joint. In accordance with some embodiments, the disclosed surgical device may be utilized in local anesthetic nerve block procedures.

The disclosed surgical device may be used, in accordance with some embodiments, to remove a target tissue mass precisely, efficiently, and without excessive bleeding at the surgical site. Moreover, in at least some cases, the surgical device may be used to determine (e.g., via the ultrasound probe) whether there has been a collection of blood at the surgical site and thus help to avoid need for subsequent surgical intervention to address such blood collection. Thus, the disclosed surgical device may facilitate biopsy surgical procedures, providing improved results as compared to traditional approaches, which have a high risk of disfigurement (from excising too much local tissue) or often require multiple surgeries (from excising an insufficient amount of local tissue). Additionally, the disclosed surgical device may be configured to reduce the opportunity for any ultrasound interference which might otherwise exist in operation of the ultrasound probe and any ultrasound surgical instrument(s) hosted by the surgical device.

System Architecture and Operation

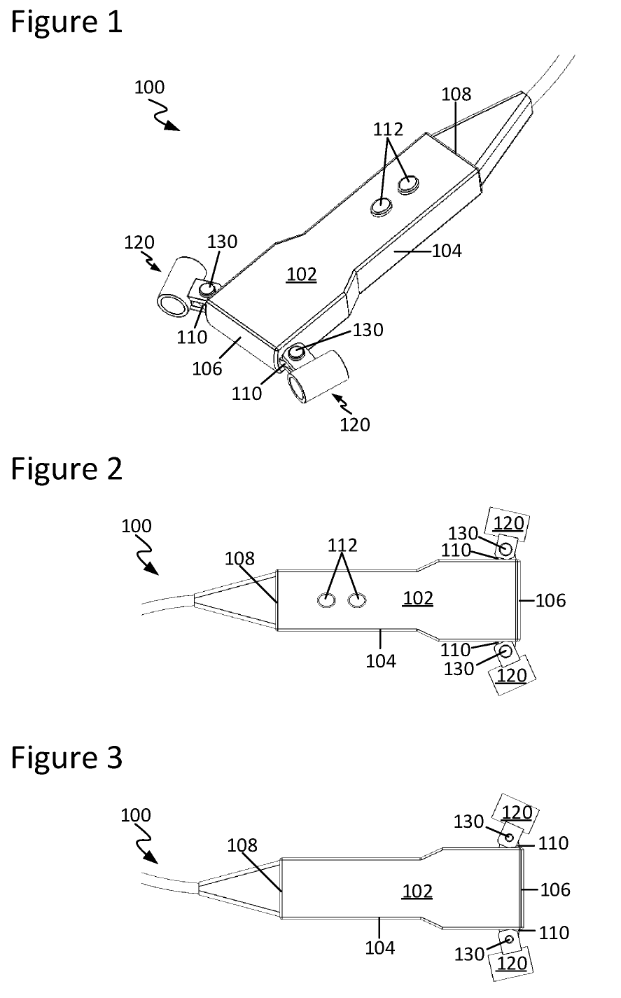

FIG. 1 illustrates an isometric view of a surgical device 100 configured in accordance with an embodiment of the present disclosure. FIGS. 2-3 illustrate top-down and bottom-up plan views, respectively, of surgical device 100 of FIG. 1. As can be seen from these figures and as discussed in further detail below, surgical device 100 may include an ultrasound probe 102 and one or more guide portions 120 configured to host any of a wide range of surgical instruments. In accordance with some embodiments, movement of a given guide portion 120 and/or a given surgical instrument hosted thereby may help to ensure that the surgical instrument remains at least partially disposed within the field-of-view (FOV) of ultrasound probe 102, and thus visible to ultrasound probe 102, in operation of surgical device 100.

Ultrasound probe 102 may be configured, in accordance with some embodiments, to transmit and/or receive ultrasound signal(s) for ultrasound imaging in operation of surgical device 100. To such ends, ultrasound probe 102 may be any suitable type of ultrasound probe, and in at least some cases may be an ultrasonic transducer having either (or both) ultrasound transmitter and receiver capabilities (e.g., transceiver capabilities), configured as typically done. Body portion 104 of ultrasound probe 102 may be configured to be held by a user's hand, a mechanical interface, or other desired device retention means suitable for use in the context of a surgical process. To this end, body portion 104 may be of sufficient dimensions and geometry to be generally ergonomic to a user's hand, securely mountable to a piece of equipment, or maneuverable as otherwise desired. Signal(s) emitted and/or intercepted by ultrasound probe 102 may pass through a head portion 106 disposed at an end of body portion 104, at least in some embodiments. Ultrasound probe 102 may be a linear probe or a curved probe, and its head portion 106 may be adapted accordingly.

Ultrasound probe 102 may be configured, in accordance with some embodiments, to provide two-dimensional and/or three-dimensional ultrasound imaging of elements within its FOV, including any surgical instrument hosted by surgical device 100, as well as any target tissue mass within its FOV. Ultrasound probe 102 may be configured, in accordance with some embodiments, to be operatively coupled with an ultrasound imaging display element 300 (discussed below), whether native or remote to surgical device 100. As ultrasound probe 102 may detect the positioning and movement of a given surgical instrument (associated with surgical device 100) within its FOV and have its imaging output displayed via ultrasound imaging display element 300, ultrasound probe 102 may be utilized in guiding operation of surgical device 100, in accordance with some embodiments. Similarly, ultrasound probe 102 may be configured, in accordance with some embodiments, to provide for determining the location (e.g., positioning within a host body), physical dimensions (e.g., depth, width, and length), or other desired characteristic(s) about a target tissue mass within its FOV.

The specific ultrasound frequency range at which ultrasound probe 102 may operate may be customized, as desired for a given target application or end-use. In some instances, the specific ultrasound imaging capabilities of ultrasound probe 102 may be customized, for example, based on the dimensions (e.g., depth and width) of the target tissue mass or other parameters of the surgical procedure to be carried out using surgical device 100. In some embodiments, ultrasound probe 102 may be configured to scan tissue to a depth in the range of about 0.1-2.0 cm (e.g., about 0.1-1.0 cm, about 1.0-2.0 cm, or any other sub-range in the range of about 0.1-2.0 cm). In some other embodiments, ultrasound probe 102 may be configured to scan tissue to a depth in the range of about 2.0-6.0 cm (e.g., about 2.0-4.0 cm, about 4.0-6.0 cm, or any other sub-range in the range of about 2.0-6.0 cm). In some other embodiments, ultrasound probe 102 may be configured to scan tissue to a depth in the range of about 6.0 cm or greater (e.g., about 8.0 cm or greater, about 10.0 cm or greater, about 12.0 cm or greater, or any other sub-range in the range of about 6.0 cm or greater).

Ultrasound probe 102 may include a connector port 108, which may be disposed at a proximal end or elsewhere along body portion 104. Connector port 108 may be configured to receive and retain, in a permanent or temporary manner, a power supply and/or control cable to facilitate operation of ultrasound probe 102 and, more generally, surgical device 100. The present disclosure is not intended to be limited only to such removable cable configurations, however, as in other embodiments, surgical device 100 may include an integrated or otherwise permanently attached version of such a cable, optionally omitting connector port 108. In a more general sense, surgical device 100 may be configured for either (or both) cordless and corded operation. Through connector port 108 (or integrated cable, as the case may be), ultrasound probe 102 may receive and/or transmit one or more signals, including electrical power and control signals, among other options. To these ends, connector port 108 may be configured, in accordance with some embodiments, to be operatively coupled with any of an ultrasound imaging display element 300, an electrosurgical power supply and control unit 400, an ultrasound power supply and control unit 500, and a foot pedal control 600, each discussed below. Other suitable configurations for connector port 108 will depend on a given application and will be apparent in light of this disclosure.

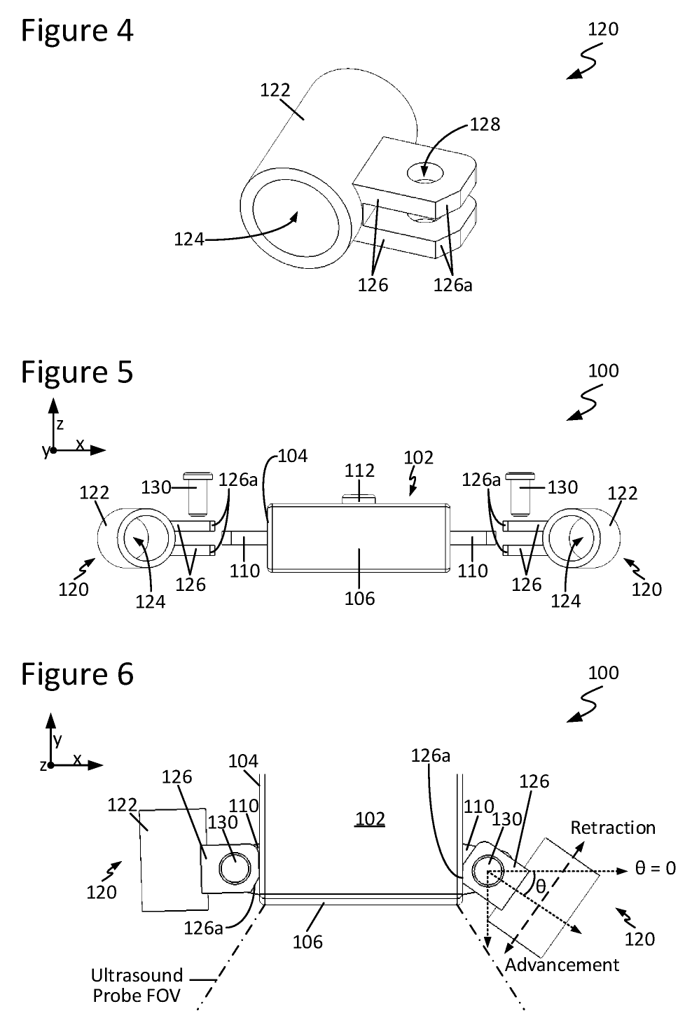

As previously noted, surgical device 100 further may include one or more guide portions 120 configured to host a given surgical instrument, in accordance with some embodiments. FIG. 4 illustrates an isometric view of a guide portion 120 configured in accordance with an embodiment of the present disclosure. FIG. 5 illustrates a partially exploded end view of a surgical device 100 including several guide portions 120, in accordance with an embodiment of the present disclosure. In some embodiments, a given guide portion 120 may be a separate element affixable to ultrasound probe 102 in a temporary or permanent manner. In some other embodiments, however, a given guide portion 120 may be formed integrally, for example, with a housing or other exterior portion of ultrasound probe 102 or a separate add-on to be operatively coupled with ultrasound probe 102.

As can be seen from FIGS. 4-5, a given guide portion 120 may include a body portion 122. In some embodiments, body portion 122 may be of generally tubular configuration, having a hollow inner bore 124. In some cases, body portion 122 may be generally cylindrical in shape (e.g., as in FIG. 4), though in some other cases, it may be generally prismatic (e.g., box-like) in shape. In a more general sense, and in accordance with some embodiments, body portion 122 may be of any desired polygonal (e.g., triangular, quadrilateral, pentagonal, hexagonal, and so forth) or curvilinear (e.g., circular, elliptical, and so forth) cross-sectional geometry, as desired for a given target application or end-use.

As can be seen further, body portion 122 may include one or more tab portions 126 extending therefrom and configured to be attached to a given mounting portion 110 of ultrasound probe 110. To that end, a given tab portion 126 may have one or more apertures 128 defined therein and configured to receive a retention pin 130 (or other retention element), in a temporary or permanent manner, which may be coupled with a given mounting portion 110. In some instances, tab portion(s) 126 may be detachable in a manner which provides for quick and easy replacement or exchange of an associated guide portion 120 in customizing the configuration of surgical device 100. In a more general sense, a given guide portion 120 may be configured, at least in some cases, as a plug-and-play element that lends itself to quick and easy changing out in the use of surgical device 100.

A given tab portion 126 optionally may include a chamfer 126a (or other tapered region) disposed at its end to be incident with the exterior of ultrasound probe 102 (or other intervening structure). As discussed in further detail below, chamfer 126a may be configured to serve as a physical stopping point in adjustment of the angular orientation of an associated guide portion 120. The dimensions, angle(s), and faceting of optional chamfer 126a may be customized to achieve a target pivot angle (θ) (see FIG. 6) and thus facilitate a given desired range of motion for an associated guide portion 120. In an example case, a given chamfer 126a may permit an associated guide portion 120 to rotate through a pivot angle θ of about 45°, though chamfers 126a of other configurations may provide for greater or lesser pivot angles θ, as desired.

A given guide portion 120 may be a substantially rigid body capable of hosting and controlling manipulation of a given surgical instrument for performing a given surgical procedure via surgical device 100. In some embodiments, a given guide portion 120 may be of monolithic construction (e.g., may constitute a single-piece element), though in some other embodiments, it may be of polylithic construction (e.g., may constitute multiple pieces operatively coupled with one another). Moreover, the dimensions and geometry of a given guide portion 120 may be customized, as desired for a given target application or end-use. In some cases, surgical device 100 may include a plurality of guide portions 120, all (or some sub-set) of which may be substantially similarly dimensioned. In some other cases, however, guide portions 120 of different dimensions may be provided for surgical device 100. A given guide portion 120 may be fixed or adjustable in one or more of its dimensions (e.g., inner diameter/width, length, etc.), in accordance with some embodiments.

In accordance with some embodiments, a given guide portion 120 may be configured to control linear movement of a given surgical instrument hosted thereby. For instance, consider FIG. 6, which illustrates a partial top-down view of a surgical device 100 including several guide portions 120, in accordance with an embodiment of the present disclosure. As generally can be seen, a given guide portion 120 may be configured to permit advancement and/or retraction of a given surgical instrument within a given range of linear movement, thereby controlling the proximal and distal movement of the surgical instrument. In this manner, a surgical instrument may be extended into or removed from the FOV of ultrasound probe 102, as well as moved closer to or farther from the surgical site, as desired. The range of linear motion may be customized and, at least in some instances, may depend on the specific configuration of the surgical instrument.

In accordance with some embodiments, a given guide portion 120 may be configured to control angular movement of a surgical instrument hosted thereby. As generally can be seen from FIG. 6, a given guide portion 120 may be adjustable in its angular orientation with respect to ultrasound probe 102 in one or more directions, thereby controlling the angular movement of the surgical instrument. To that end, a given guide portion 120 may pivot about a retention pin 130 (or other retention element) operatively coupling it to a mounting portion 110, in accordance with some embodiments. In some embodiments, a given guide portion 120 may have a biaxial (e.g., planar) range of angular movement, though in some other embodiments, triaxial movement may be provided.

As previously noted, a given guide portion 120 may be configured to rotate through a target pivot angle θ, which may be customized as desired. In some cases, pivot angle θ may be about 45° (e.g., ±5°), though in other cases, pivot angles greater than or less than 45° may be provided (e.g., about 30° or less, about 15° or less, about 60° or more, or any other desired range greater than or less than 45°). Also, as previously noted, an optional chamfer 126a may provide a hard stop for the range of angular movement of an associated guide portion 120 (and thus a surgical instrument hosted thereby). Once within a given target orientation, a given guide portion 120 may be locked or otherwise fixed in position to maintain such orientation temporarily or permanently.

In accordance with some embodiments, by virtue of a given retention pin 130 serving as a pivot point and/or the interfacing of optional chamfer 126a with the exterior of ultrasound probe 102 (or other intervening surface), an associated guide portion 120 may have a range of defined pivoting articulation with respect to ultrasound probe 102. A given guide portion 120 may be adjusted such that a surgical instrument hosted thereby may be oriented substantially parallel to a length of ultrasound probe 102 (e.g., the condition of pivot angle θ being substantially equal to zero). A given guide portion 120 may be adjusted such that a surgical instrument hosted thereby may be oriented at an acute or obtuse angle with respect to a length of ultrasound probe 102 (e.g., the condition of pivot angle θ being greater than or less than zero).

A given guide portion 120 may be disposed on any surface (e.g., side, top/dorsal, bottom/ventral, or other) of body portion 104 or head portion 106 of ultrasound probe 102. In some embodiments, such as that generally shown in FIGS. 1-3, surgical device 100 may include two guide portions 120 disposed substantially opposite one another across body portion 104 of ultrasound probe 102. However, the present disclosure is not intended to be so limited, as in accordance with some other embodiments, surgical device 100 may include fewer (e.g., one) or more (e.g., three, four, five, or more) guide portions 120 arranged as desired. In accordance with some embodiments, a given guide portion 120 may be disposed on ultrasound probe 102 in a manner which optimizes or otherwise improves visualization by ultrasound probe 102 of a surgical instrument hosted by that guide portion 120. Other suitable configurations for guide portion(s) 120 will depend on a given application and will be apparent in light of this disclosure.

In accordance with some embodiments, surgical device 100 may be configured such that its guide portion(s) 120 may be adjustable to maximize or otherwise customize visualization by ultrasound probe 102 of a given surgical instrument hosted by such guide portion(s) 120. In accordance with some embodiments, a given guide portion 120 may be configured to ensure that a given surgical instrument is maintained in a centered or other desired disposition with respect to the FOV of ultrasound probe 102. A given guide portion 120 may be adjustable to help ensure that an attendant surgical instrument is fully or otherwise sufficiently disposed proximate a target tissue mass, thereby facilitating removal of or other operation on the entire target tissue mass and, at least in some instances, realizing improvements in efficiency of tissue removal as compared to existing devices. Adjustment of the orientation, dimensions, or other aspect(s) of a given guide portion 120 may be provided by any suitable mechanical, electronic, or manual means, as will be apparent in light of this disclosure, and may be performed automatically or upon instruction or manipulation by a user or other external control source, in accordance with some embodiments.

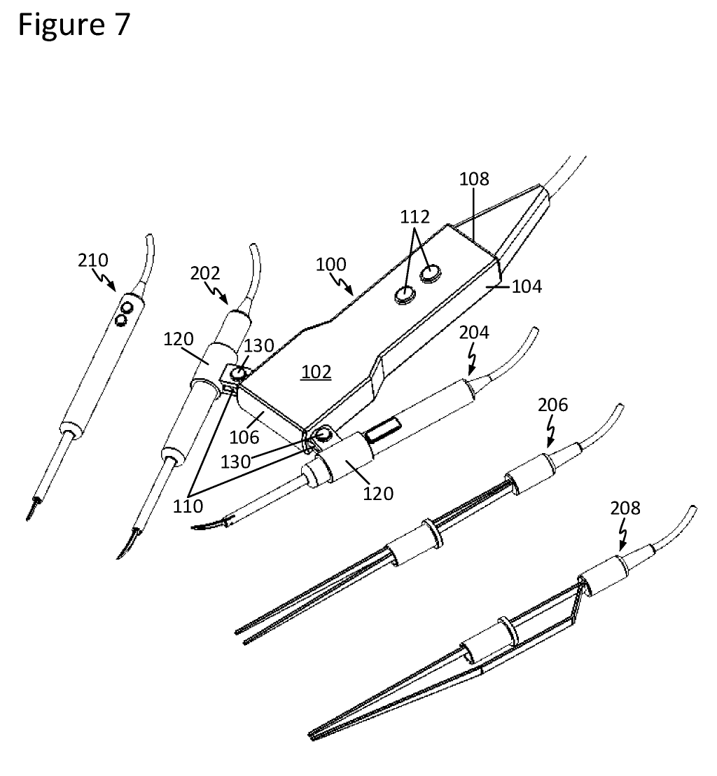

In accordance with some embodiments, surgical device 100 may be configured to host any of a wide range of surgical instruments under the control of a user or other suitable control system of surgical device 100. A given surgical instrument may be inserted within, attached to, or otherwise operatively coupled with a given guide portion 120, in a permanent or temporary manner, and permitted to slide, rotate, flex, bend, or otherwise articulate relative to its host guide portion 120, in accordance with some embodiments. In cases where a given surgical instrument may be detachable from a given guide portion 120, it may be considered, in a general sense, a replaceable component of surgical device 100. In some embodiments, a given guide portion 120 may be adjustable in its configuration, providing a given degree of universality, which can accommodate any of a multitude of different surgical instruments. In some other embodiments, however, a given guide portion 120 may be specifically configured to accommodate only a single surgical instrument or limited grouping of surgical instruments, providing a more dedicated operability. To facilitate detection by ultrasound probe 102, it may be desirable, at least in some instances, to ensure that a given surgical instrument is echogenic, in part or in whole. FIG. 7 illustrates an isometric view of a surgical device 100 with an assortment of surgical instruments which it may be configured to host, in accordance with some embodiments of the present disclosure.

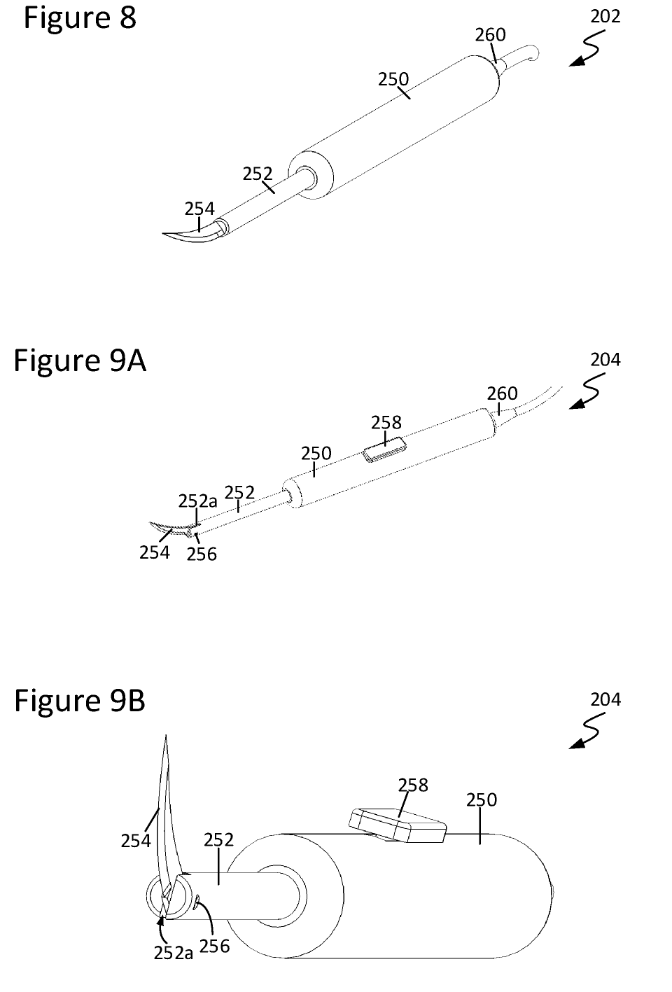

In accordance with some embodiments, surgical device 100 may be configured to host one or more electrosurgical instruments. For instance, consider FIG. 8, which illustrates an isometric view of a straight monopolar electrosurgical knife 202 configured in accordance with an embodiment of the present disclosure. Further consider FIGS. 9A-9B, which illustrate several isometric views of an articulated monopolar electrosurgical knife 204 configured in accordance with an embodiment of the present disclosure. As can be seen from these figures, a given electrosurgical knife 202, 204 may include a body portion 250, which may be held or otherwise controlled via a guide portion 120. With electrosurgical knife 202 (FIG. 8), a sharp surgical knife blade 254 may be affixed to or formed integrally with a distal end of a shaft portion 252 extending from body portion 250. With electrosurgical knife 204 (FIGS. 9A-9B), however, a sharp surgical knife blade 254 may be at least partially disposed in a groove 252a formed in a distal end of a shaft portion 252 extending from body portion 250, and a pin 256 may be inserted through shaft portion 252 such that surgical knife blade 254 may pivot at that point within groove 252a. To effectuate movement of surgical knife blade 254 within groove 252a, electrosurgical knife 204 may include an actuator 258, such as a sliding actuator. Movement of actuator 258 in a first direction may cause surgical knife blade 254 to pivot about pin 256 in a first direction, whereas movement in a second direction may cause pivoting about pin 256 in a second direction. The range of movement of surgical knife blade 254 may be customized, as desired for a given target application or end-use. In at least some cases, surgical knife blade 254 may be configured to pivot about pin 256 through an angle of about 10° or more, about 15° or more, about 20° or more, about 30° or more, about 45° or more, about 60° or more, or about 90° or more. In some other cases, however, pivoting through an angle of about 10° or less may be provided.

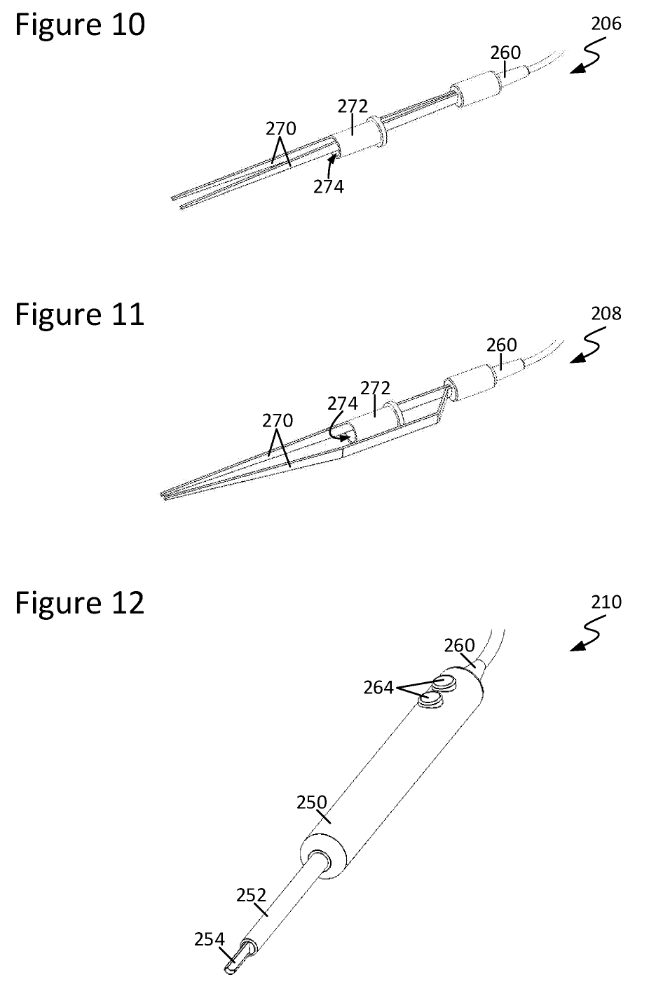

FIGS. 10-11 illustrate an isometric view of narrow-grip bipolar electrosurgical forceps 206 and wide-grip bipolar electrosurgical forceps 208 configured in accordance with some embodiments of the present disclosure. As can be seen from these figures, a given electrosurgical forceps 206, 208 may include two tip portions 270 disposed opposite one another and configured to be manipulated toward and away from one another, as typically done. Also, an insert portion 272 may be provided at one of the tip portions 270 and configured to be held or otherwise controlled via a guide portion 120. In some embodiments, insert portion 272 and a tip portion 270 optionally may be of monolithic construction (e.g., may constitute a single element), though other embodiments may be of polylithic construction (e.g., may constitute multiple elements operatively coupled with one another). With electrosurgical forceps 206 (FIG. 10), both tip portions 270 may be disposed within inner bore 274 of insert portion 272. With electrosurgical forceps 208 (FIG. 11), however, one tip portion 270 may be disposed within inner bore 274 of insert portion 272, and the other tip portion 270 may be disposed outside of inner bore 274.

A given electrosurgical knife 202, 204 or electrosurgical forceps 206, 208 may include a connector 260 configured to be operatively coupled with either (or both) an electrosurgical power supply and control unit 400 and a foot pedal control 600 (each discussed below). In this manner, a given electrosurgical instrument may be provided with one or more electrosurgical functions, such as cutting, coagulating, and cauterizing, for example, while hosted by a given guide portion 120 of surgical device 100.

In accordance with some embodiments, surgical device 100 may be configured to host one or more ultrasound surgical instruments. For instance, consider FIG. 12, which illustrates an isometric view of an ultrasound straight knife 210 configured in accordance with an embodiment of the present disclosure. As can be seen, ultrasound straight knife 210 may include a body portion 250, which may be held or otherwise controlled via a guide portion 120. A sharp surgical blade 254 may be affixed to or formed integrally with a distal end of a shaft portion 252 extending from body portion 250. Ultrasound straight knife 210 may include a connector 260 configured to be operatively coupled with either (or both) an ultrasound power supply and control unit 500 and a foot pedal control 600 (each discussed below). In this manner, ultrasound straight knife 210 may be provided with one or more ultrasound surgical functions, such as cutting, coagulating, and cauterizing, for example, while hosted by a given guide portion 120 of surgical device 100. Ultrasound straight knife 210 may include one or more control features 264 for selection between its operational modalities and which may be of any of the example configurations discussed below, for instance, with respect to control(s) 112. Other suitable configurations for ultrasound straight knife 210 will depend on a given application and will be apparent in light of this disclosure.

In accordance with some embodiments, surgical device 100 may be configured to host ultrasound forceps. Such ultrasound forceps may be configured to be held or otherwise controlled via a guide portion 120. The ultrasound forceps may include a plurality of tip portions configured to be manipulated toward and away from one another, as typically done. In some instances, the ultrasound forceps may include a jaw-like arrangement of tip portions. The ultrasound forceps may be provided with any one or combination of the various example ultrasound surgical functions discussed above, for instance, with respect to ultrasound straight knife 210, in accordance with some embodiments. Other suitable configurations for the ultrasound forceps or any other desired ultrasound surgical instrument will depend on a given application and will be apparent in light of this disclosure.

The present disclosure is not intended to be limited only to electrosurgical and ultrasound surgical instruments, as other types of instruments typically used in surgeries (e.g., laser-based surgical instruments, needles, suction-based instruments, or tissue retractor instruments, among others) may be utilized in conjunction with surgical device 100, in accordance with some other embodiments. Moreover, the present disclosure is not intended to be limited only to cut and coagulation functions, as other surgical modalities, such as, for example, cauterizing, grabbing, severing, injecting, withdrawing, coring, and suctioning, among others, may be provided via surgical instruments compatible for use with surgical device 100, in accordance with some other embodiments.

In accordance with some embodiments, surgical device 100 may include one or more controls 112 configured to control operation of ultrasound probe 102 and/or a given surgical instrument hosted by surgical device 100. Control may be provided via mechanical, electronic, or other suitable means and may be customized, as desired for a given target application or end-use. A given control 112 may be operable by a user (or host piece of equipment or other external control element) via a physical control feature (e.g., such as a physical button, switch, knob, pressure sensor, toggle, slider, and so forth) or a virtual control feature (e.g., a touch-sensitive icon or other element providing any one or more of the aforementioned physical control feature functionalities) provided on surgical device 100.

In accordance with some embodiments, control(s) 112 may be configured to control the surgical modalities of surgical device 100. In some cases, surgical device 100 may include a control 112 for causing a given surgical instrument to perform any one, or combination, of a cutting action, a coagulation action, a cauterizing action, and a grabbing action during a surgical procedure. Other suitable control(s) 112 will depend on a given application and will be apparent in light of this disclosure.

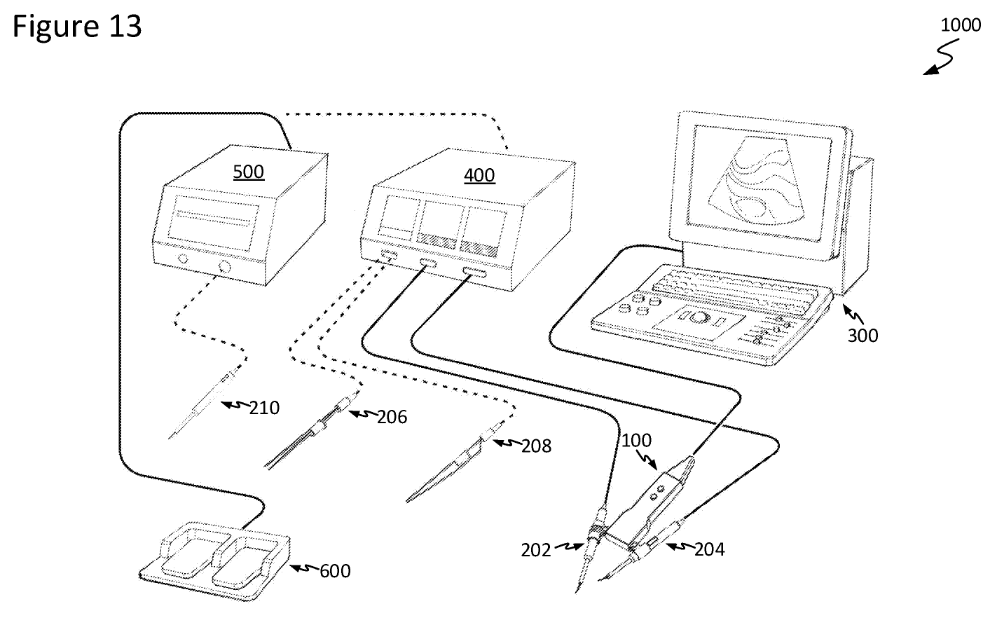

FIG. 13 illustrates an example surgical system 1000 configured in accordance with an embodiment of the present disclosure. As can be seen, system 1000 may include any of a wide range of optional power supply and control elements that may be utilized in operation of surgical device 100 and the one or more surgical instruments hosted thereby, as previously discussed. Surgical device 100 may be configured to utilize either (or both) a native power supply and an external power supply. As previously noted, ultrasound probe 102 may include a connector port 108 configured to be operatively coupled with an external power source. In some embodiments, surgical device 100 additionally (or alternatively) may include a native (e.g., on-board) power supply element, such as a battery or other energy storage device, which may be permanently integrated with or replaceable within surgical device 100. In some cases, surgical device 100 may include either (or both) of an on-board power supply (e.g., a battery, integrated or removable) and controller means. In some instances, a given controller may be configured with one or more controls to facilitate performance of any of the various surgical actions (discussed above) utilizing surgical device 100. Numerous suitable configurations and variations will be apparent in light of this disclosure.

In accordance with some embodiments, system 1000 optionally may include an ultrasound imaging display element 300, which may be configured to display an image based on ultrasound imaging data received from an ultrasound probe 102 operatively coupled, directly or indirectly, therewith. To that end, display element 300 may be any electronic visual display (EVD) or other display device configured to display or otherwise generate an image (e.g., image, video, text, or other displayable content) thereat. In some cases, display element 300 optionally may be a touchscreen display or other touch-sensitive surface. In some embodiments, display element 300 may be a stand-alone component external to surgical device 100 and configured to communicate therewith using any suitable wired or wireless (or both) communication means. In some other embodiments, however, display element 300 may be integrated, in part or in whole, with surgical device 100. In some such cases, display element 300 may be operatively coupled to ultrasound probe 102 via an arm, pivot, or other suitable connection means, which optionally may permit display element 300 to be rotatable with respect to body portion 104 of ultrasound probe 102. In a more general sense, display element 300 may be native to surgical device 100 or a remote device operatively coupled therewith. In operation of surgical device 100, ultrasound probe 102 may deliver an ultrasound imaging signal to display element 300, which in turn may display an image (or other data) based on such signal. Other suitable configurations for optional ultrasound imaging display element 300 will depend on a given application and will be apparent in light of this disclosure.

In accordance with some embodiments, system 1000 also may include an electrosurgical power supply and control unit 400, which may be configured to provide power and/or control to a given electrosurgical element operatively coupled therewith. In an example embodiment, electrosurgical unit 400 may be a BOVIE® electrosurgical unit, configured as typically done. Electrosurgical unit 400 may be configured for either (or both) monopolar or bipolar output for use in performing electrosurgery via surgical device 100, and any one (or combination) of electrosurgical knives 202, 204 and electrosurgical forceps 206, 208 may be operatively coupled with electrosurgical unit 400, in accordance with some embodiments.

In accordance with some embodiments, system 1000 also may include an ultrasound surgical power supply and control unit 500, which may be configured to provide power and/or control to a given ultrasound surgical element operatively coupled therewith. Ultrasound surgical unit 500 may be configured for ultrasound output for use in performing ultrasonic surgery via surgical device 100, and straight ultrasound knife 210 may be operatively coupled with ultrasound surgical unit 500, in accordance with some embodiments.

In accordance with some embodiments, system 1000 optionally further may include a foot pedal control 600, which may be operatively coupled, directly or indirectly, with any (or all) of ultrasound imaging display element 300, electrosurgical unit 400, ultrasound surgical unit 500, and surgical device 100. In some embodiments, foot pedal control 600 may be configured to provide control to a given surgical element operatively coupled therewith via one or more pedals. In an example case, foot pedal control 600 may include a first pedal associated with a cut function and a second pedal associated with a coagulation function of a given surgical instrument. In such case, by activating the first pedal, the cut function may be engaged/disengaged, and by activating the second pedal, the coagulation function may be engaged/disengaged. It should be noted, however, that the foot pedal control 600 is not intended to be limited only to these two functions, as in a more general sense, any desired function may be associated with a given pedal, as desired for a given target application or end-use. In some instances, a given pedal may be user-assignable to a given function.

Ultrasound Interference Management

When using multiple ultrasound-based surgical elements simultaneously in proximity to one another, the ultrasound signal output by one such element can cause interference with the ultrasound input of another such element, at least in some cases. For example, in using an ultrasound surgical instrument in conjunction with an ultrasound probe, the ultrasonic frequency of the surgical instrument may interfere with the ultrasound readings performed by the ultrasound probe. This can lead to unreliable ultrasound imaging, which can have undesirable consequences in the course of surgery.

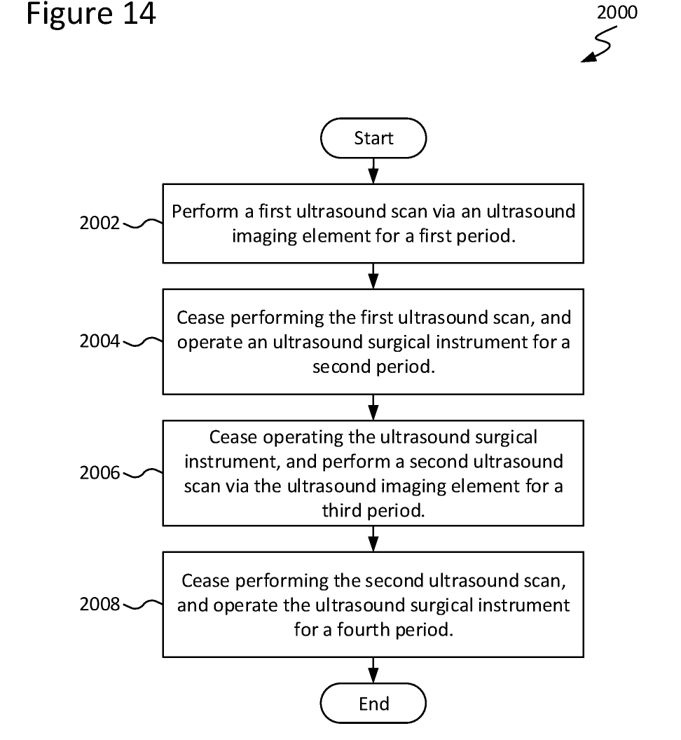

Thus, and in accordance with some embodiments, surgical device 100 may be configured to prevent or otherwise reduce such ultrasound interference complications. FIG. 14 is a flow chart illustrating a method 2000 of reducing ultrasound interference in use of a surgical device, in accordance with an embodiment of the present disclosure. Method 2000 may begin as in block 2002 with performing a first ultrasound scan via an ultrasound imaging element for a first period. In an example case, the ultrasound imaging element may be ultrasound probe 102, discussed above. In an example case, the first period may be about 1 sec or less in duration, although longer or shorter periods may be provided, as desired for a given target application or end-use. Method 2000 may continue as in block 2004 with ceasing performing the first ultrasound scan, and operating an ultrasound surgical instrument for a second period. In an example case, the ultrasound surgical instrument may be ultrasound straight knife 210, discussed above, or any other ultrasound surgical instrument compatible for use with surgical device 100, as will be apparent in light of this disclosure. Method 2000 may continue as in block 2006 with ceasing operating the ultrasound surgical instrument, and performing a second ultrasound scan via the ultrasound imaging element for a third period. In an example case, the third period may be about the same in duration as the first period, though it is not required to be. Method 2000 may continue as in block 2008 with ceasing performing the second ultrasound scan, and operating the ultrasound surgical instrument for a fourth period. As will be appreciated in light of this disclosure, any (or all) of the various actions of blocks 2002-2008 may be performed at any time, in any order, and any number of times, as desired for a given target application or end-use.

In being configured to implement method 2000, in part or in whole, surgical device 100 may be configured to intermittently perform an ultrasound scan via ultrasound probe 102 for a first period of desired duration and then allow for operation of ultrasound straight knife 210 (or other ultrasound-based surgical instrument hosted by surgical device 100) for a second period of desired duration. At the user's or controller's direction, or after some predetermined period has elapsed, ultrasound probe 102 again may perform a scan, and the ultrasound surgical instrument may remain idle in operation during such time. Once the scan is completed, the ultrasound surgical instrument again may be operable, while the ultrasound probe 102 remains idle in operation during such time. In this manner, operation of ultrasound probe 102 and of a given ultrasound surgical instrument may not overlap (or may overlap only negligibly), obviating ultrasound interference complications which otherwise might manifest in utilizing multiple ultrasound-based devices simultaneously. In at least some embodiments, surgical device 100 may be provided with an override feature which allows for immediate cessation of a scanning process so that the ultrasound-based surgical instrument may be used whenever desired. In at least some instances, this may help to ensure absolute control over the timely operation of surgical device 100 and the safety of the surgical patient.

As will be appreciated in light of this disclosure, method 2000 may be provided for surgical device 100 via either (or both) hardware and software solutions. In accordance with some embodiments, surgical device 100 (or other element of system 1000 more generally) may include one or more computing and communication elements (e.g., a processor, memory, a communication bus, a communication module, and so forth) which may be utilized in storing and accessing data related to, as well as carrying out executable code associated with, any of the various actions of method 2000 in operation of surgical device 100.

Alternatively, or in addition to method 2000, surgical device 100 may be configured such that its ultrasound probe 102 and any hosted ultrasound surgical instrument (e.g., such as ultrasound straight knife 210 or the ultrasound forceps discussed above) operate at the same (or substantially similar, within a given tolerance) ultrasound wavelength(s). As will be appreciated in light of this disclosure, in at least some instances, this may help to eliminate or otherwise render negligible the opportunity for ultrasound interference in the course of operating a surgical device 100 including multiple ultrasound-based elements.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present invention be limited not by this detailed description, but rather by the claims appended hereto. Future-filed applications claiming priority to this application may claim the disclosed subject matter in a different manner and generally may include any set of one or more limitations as variously disclosed or otherwise demonstrated herein.

1. A surgical device comprising:
an ultrasound probe; and
at least one guide portion comprising:
a tubular body portion having a hollow inner bore defined therein and configured to host a surgical instrument; and
a tab portion extending from the body portion and configured to be mounted to an exterior of the ultrasound probe via a retention pin, wherein the tab portion includes a chamfer configured to be incident with an exterior of the ultrasound probe;
wherein the at least one guide portion is configured to permit:
linear movement of the surgical instrument with respect to the at least one guide portion; and
angular movement of the surgical instrument with respect to the ultrasound probe, with the chamfer being configured to provide a stopping point for the angular movement when incident with the exterior of the ultrasound probe.

2. The surgical device of claim 1, wherein the at least one guide portion comprises two guide portions, one of which is disposed at a first side of the ultrasound probe, and the other of which is disposed at an opposing second side of the ultrasound probe.

3. The surgical device of claim 2, wherein the two guide portions are dissimilar in at least one dimension.

4. The surgical device of claim 1, wherein the angular movement of the surgical instrument with respect to the ultrasound probe is planar.

5. The surgical device of claim 1, wherein the angular movement of the surgical instrument with respect to the ultrasound probe is triaxial.

6. The surgical device of claim 1, wherein the tubular body portion is adjustable in at least one dimension.

7. The surgical device of claim 1, wherein the guide portion is of monolithic construction.

8. The surgical device of claim 1, wherein in being configured to host the surgical instrument, the guide portion is configured to host at least one of an electrosurgical knife, an articulated electrosurgical knife, electrosurgical forceps, an ultrasound knife, and ultrasound forceps.

9. The surgical device of claim 1, further comprising at least one control feature provided on the ultrasound probe and configured to control a surgical modality of the surgical instrument hosted by the guide portion.

10. The surgical device of claim 1, further comprising at least one of:
an integrated battery; and
a removable battery.

11. The surgical device of claim 1, further comprising a display element configured to display imaging data received from the ultrasound probe.

12. A surgical system comprising:
the surgical device of claim 1; and
a power supply and control unit configured to be operatively coupled with the surgical device.

13. The surgical system of claim 12, wherein the power supply and control unit is an electrosurgical power supply and control unit.

14. The surgical system of claim 12, wherein the power supply and control unit is an ultrasound power supply and control unit.

15. The surgical system of claim 12, further comprising a foot pedal control configured to:
be operatively coupled with at least one of the surgical device and the power supply and control unit; and
control a surgical modality of the surgical instrument hosted by the guide portion of the surgical device.

16. The surgical system of claim 12, further comprising an ultrasound imaging display device configured to be operatively coupled with the surgical device and to display imaging data received from the ultrasound probe of the surgical device.

17-20. (canceled)
